(19) United States
(12) Patent Application Publication
(10) Pub. No.: US 2023/0298729 A1
(43) Pub. Date: Sep. 21, 2023

(54) Meal Plan Creation Systems and Methods

(71) Applicant: Eat This Much, Inc., Lawndale, CA (US)

(72) Inventor: Louis Thomas DeMenthon, Lawndale, CA (US)

(21) Appl. No.: 17/695,675

(22) Filed: Mar. 15, 2022

(51) Int. Cl. G16H 20/60; G16H 40/67

(52) U.S. Cl. CPC G16H 20/60; G16H 40/67

(57) ABSTRACT

Embodiments of the inventive subject matter are directed to meal planning systems and methods. Meal planning services that implement the inventive subject matter use a platform server (e.g., cloud services) that makes it possible for users to access the service via user device (e.g., mobile phone). Users can input an array of different parameters including meal preferences, dietary restrictions, body attributes, and so on. The platform server uses information collected from a user to provide meal suggestions to the user. Additionally, users can specify that certain meals should be set up as placeholders at the time of meal plan creation. In some embodiments, a user can replace one or more existing meals in a meal plan with a placeholder meal. Placeholder meals have nutritional attributes (e.g., calories, fat, protein, carbohydrates) and can allow flexibility for users to stick to a diet without sticking to specifically selected meals at all times.

### FIELD OF THE INVENTION

The field of the invention is meal planning implemented as an online service.

### BACKGROUND

The background description includes information that may be useful in understanding the present invention. It is not an admission that any of the information provided in this application is prior art or relevant to the presently claimed invention, or that any publication specifically or implicitly referenced is prior art.

Many diets, boiled down, are ways for people to get a better hold on nutritional and caloric input into their bodies. Some diets are directed more to calorie counting, while others take a more integrated approach by focusing more on overall nutritional value of food. Diets, therefore, help people plan their meals so they can eat healthy and meet their nutritional and caloric goals.

In the past, diets were something people just wrote down and followed. But with modern technology, it has never been easier for people to create entire meal plans that map out multiple meals every day over the course of weeks, months, or an indeterminate amount of time for those that intend to maintain a diet. Past efforts to implement meal planning software, though, have failed to maintain flexibility that helps people stick to their meal plans. For example, it's not always possible to eat every meal as planned, every single day. And when people miss meals, or don't eat a meal according to their meal plan, that can leave them feeling dejected or with feelings of failure. Similarly, sometimes people just cannot know what they will be eating in certain settings (e.g., a work lunch).

By allowing a user to change a meal in their meal plan into a placeholder meal that has attributes of a typical meal for that user, but does not specify any particular food or recipe, the user can still meet their dietary goals without causing the user to feel as though they are failing to adhere to their meal plan. Thus, there is still a need in the art for a meal planning platform that allows users to add placeholder meals for added flexibility in their meal plan.

### SUMMARY OF THE INVENTION

The present invention provides apparatuses, systems, and methods directed to meal planning. In one aspect of the inventive subject matter, a method of generating a meal plan is contemplated, the method comprising: receiving, at a platform server from a user device, meal plan parameters comprising placeholder meal parameters for at least one meal; generating, by the platform server, a meal plan according to the meal plan attributes, the meal plan having a first meal and a second meal where the second meal comprises a placeholder meal having attributes according to the placeholder meal parameters; and making the meal plan accessible to the user device.

In some embodiments, at least one placeholder meal parameter includes a specified meal slot, where the specified meal slot is defined by the user via the user device. This information is used to determine which meal will be replaced with the placeholder meal. The placeholder meal parameters can include quantities for calories, fat, protein, and carbohydrates. In some embodiments, the meal plan parameters include daily quantities for calories, fat, protein, and carbohydrates. Quantities of calories, fat, protein, and carbohydrates of the placeholder meal can then be determined according to the daily quantities for calories, fat, protein, and carbohydrates (e.g., by dividing the daily quantities by a number of meals, which can include weighting based on meal type).

In another aspect of the inventive subject matter, a method of generating a meal plan is contemplated, the method comprising: receiving, at a platform server from a user device, meal plan parameters; generating, by the platform server, a meal plan according to the meal plan parameters, the meal plan having a first meal and a second meal; making the meal plan accessible to the user device; receiving, at the platform server from the user device, instructions to convert the second meal to a placeholder meal; generating, by the platform server, an updated meal plan comprising the placeholder meal; and making the updated meal plan accessible to the user device.

In some embodiments, the instructions include a specified meal slot, where the specified meal slot is defined by the user via the user device and indicates that the second meal should be replaced with the placeholder meal. The placeholder meal parameters can include quantities for calories, fat, protein, and carbohydrates. In some embodiments, the meal plan parameters daily quantities for calories, fat, protein, and carbohydrates. Quantities of calories, fat, protein, and carbohydrates of the placeholder meal can then be determined according to the daily quantities of calories, fat, protein, and carbohydrates (e.g., by dividing the daily quantities by a number of meals, which can include weighting based on meal type).

One should appreciate that the disclosed subject matter provides many advantageous technical effects including an ability to create flexible meal plans or to make existing meal plans more flexible by introducing placeholder meals that still include nutritional information.

Various objects, features, aspects, and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is a schematic of how different computing devices communicate to implement embodiments of the inventive subject matter.

FIG. 2 is a flowchart showing an embodiment where a placeholder meal is included at the time of meal plan generation.

FIG. 3 is a flowchart showing an embodiment where a placeholder meal is introduced after meal plan generation.

FIG. 4 shows an interface used to collect diet and calorie information during meal plan creation.

FIG. 5 shows an interface used to collect user profile information.

FIG. 6 shows an interface used to collect additional diet information including allergies.

FIG. 7 shows an interface that provides a user with recommended and editable nutrition targets based on the user's profile information.

FIG. 8 shows an interface used to collect food preference information that can help with automatic food recommendations.

FIG. 9 shows an interface that allows a user to edit a specific meal, including an option to make the meal into a placeholder meal.

FIG. 10 shows how the interface from FIG. 9 changes when the meal is set to a placeholder meal.

FIG. 11 shows an alternative interface that allows users to change a meal into a placeholder meal.

FIG. 12 shows an interface that presents a user with placeholder meal information.

FIG. 13 shows an interface that allows a user to view and edit their online pantry.

### DETAILED DESCRIPTION

The following discussion provides example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

As used in the description in this application and throughout the claims that follow, the meaning of “a,” “an,” and “the” includes plural reference unless the context clearly dictates otherwise. Also, as used in the description in this application, the meaning of “in” includes “in” and “on” unless the context clearly dictates otherwise.

Also, as used in this application, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

In some embodiments, the numbers expressing quantities used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements. Moreover, and unless the context dictates the contrary, all ranges set forth in this application should be interpreted as being inclusive of their endpoints and open-ended ranges should be interpreted to include only commercially practical values. Similarly, all lists of values should be considered as inclusive of intermediate values unless the context indicates the contrary.

It should be noted that any language directed to a computer or computing device should be read to include any suitable combination of computing devices, including servers, interfaces, systems, databases, agents, peers, controllers, or other types of computing devices operating individually or collectively. One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. In especially preferred embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges preferably are conducted over a packet-switched network, the Internet, LAN, WAN, VPN, or other type of packet switched network. The following description includes information that may be useful in understanding the present invention. It is not an admission that any of the information provided in this application is prior art or relevant to the presently claimed invention, or that any publication specifically or implicitly referenced is prior art.

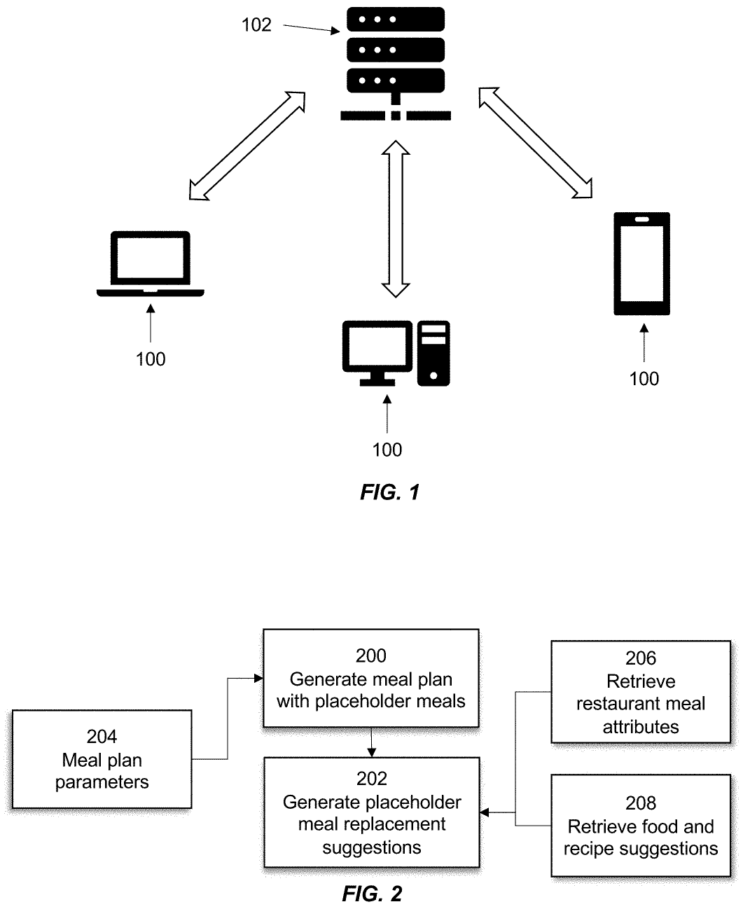

Systems and methods of the inventive subject matter are directed to meal plan creation that features a placeholder meal function. Embodiments are configured such that user devices 100 are configured to access a platform server 102 as shown in FIG. 1. Double sided arrows between the platform server 102 and user devices 100 signify network connections and do not necessarily imply direct connections, as internet traffic can be routed through any number of devices in transit between different computing devices. Throughout this application, in instances when a user is described as taking an action, it should be understood that the user takes that action via a user device, which can be any type of computing device that can access the internet (e.g., phone, laptop, computer, smart refrigerator, etc.).

The platform server has software configured to facilitate user access to meal planning software of the inventive subject matter. The term “platform server” refers to modern server technology, which can be a single computer, a set of computers working together as one, etc. Computers acting as servers do not need to be the same computers at all times so long as functions necessary for the inventive subject matter can be carried out.

FIG. 2 is a flowchart showing how a meal plan having one or more meals in one or more meal slots can be created such that at least one placeholder meal is included at the time of meal plan creation. Notably, not all steps in the flow chart must be executed in order according to their reference numbers. Step order can be interpreted by how each step is described, below, according to context and necessity of order execution. For example, step 200 must occur before step 202, but step 206 does not necessarily need to occur before step 208. A placeholder meal indicates that a user intends to eat that meal, but that the user does not yet know what that meal will be. Placeholder meals, as described below in more detail, include general details about the meal such as quantities of calories, fat, carbohydrates, and proteins, but do not give any additional detail as to what foods will be consumed.

In step 200, a meal plan is generated with placeholder meals set at time of meal plan generation. Thus, a user on a user device interacts with a platform server by, e.g., providing information sufficient for the platform server to generate a meal plan. This can be accomplished in a number of ways, including by a user device (e.g., a smart phone, tablet, computer, etc.) accessing the platform server using a web browser or software application. To generate a meal plan, the platform server needs meal planning parameters. Step 204 thus shows that meal planning parameters are sent to the platform server so that the meal plan can be generated with one or more placeholder meals included. These parameters can be at least partially user defined and sent to the platform server from a user device. Meal planning parameters can include calories per day, number of meals, number of snacks, calories for each meal, calories for each snack, meal recipes, mealtimes, nutrition targets, meal sizes (e.g., small, medium, large), diet type (e.g., no diet type, paleo, vegetarian, vegan, ketogenic, Mediterranean, Pescatarian, Zone diet, gluten-free, high protein, and so on), and so on. Although some diet types are included as examples, it should be understood that additional diet types can be implemented without departing from the inventive subject matter. Additional meal planning parameters can include a desired maximum cook time, a desired minimum cook time (which can, e.g., be expressed as “must cook” in instances where the minimum cook time is set very low such as 0-5 minutes), prep time (e.g., minimum, and maximum), daily budget (e.g., minimum and maximum).

In some embodiments, users can input body measurements (e.g., age, weight, height, body composition, etc.), which can also be considered in creating a meal plan or, in some embodiments, in making better meal suggestions for that user. Within the meal planning parameters, a user can also provide customized placeholder meal parameters to, e.g., define quantities of calories, fat, protein, and carbohydrates. Placeholder meal parameters can also include a specified meal slot that a placeholder meal will take, a time of day, a frequency, a recurrence, and so on. Thus, in some embodiments, a user can set, e.g., one or more meals to be a placeholder meal, and that placeholder meal can be set according to a user's meal plan settings (e.g., including total calories, fat, protein, and carbs per day) or that placeholder meal can be set according to a user's custom specification. Alternatively, if a user fails to provide sufficient details about a particular meal, the platform server can automatically set that meal to be a placeholder meal. For example, if a user indicates that a meal should be eaten (e.g., they include lunch in their meal plan), but they do not provide enough parameters for the platform server to find a meal for that meal slot, the meal will be set as a placeholder. In some embodiments, users can specify that they do not want a particular meal to be planned (e.g., this can be convenient during sign up to minimize how much information a user is required to enter). In such cases, placeholder meals can be set for those meal slots.

Thus, in step 200, and based on information received from the user device (e.g., information provided by the user and transmitted using the user device), the platform server creates a meal plan for the user. That meal plan is then made available to the user device. In some embodiments, the meal plan—or information sufficient to re-create the meal plan locally—is sent to the user device, where the user device can then display the meal plan to the user.

Once a meal plan has been created, the platform server can retrieve additional information relating to placeholder meals. In step 206, for example, the platform server retrieves restaurant meal attributes, and in step 208, the platform server retrieves food and recipe suggestions. Although both steps 206 and 208 are optional, these steps can facilitate meal recommendations where placeholder meals are present in a meal plan. Thus, per step 202, meal replacement suggestions that are identified according to, e.g., restaurant meal attributes, food and recipe suggestions, or both can then be sent to a user, giving the user the ability to choose a meal to substitute into a meal plan where a placeholder meal exists. As discussed above, meal replacement suggestions can be based on any attribute that is user-defined or user-selected within the platform server (e.g., dietary restrictions, previously selected meal replacements, pantry ingredients, etc.). Additionally, meal replacement suggestions can also account for a user's location (e.g., GPS, address, user-defined location, etc.), a desired search radius, specified preferred restaurants, and so on. User profile attributes such as nutrition targets, budget, cook time, etc., can also be accounted for when suggesting meal replacements. In some embodiments, instead of replacing a placeholder meal with a meal suggestion, a user can convert the placeholder meal into an empty meal (e.g., a meal with zero values for all the nutrients that can indicate that a meal has or will be skipped entirely).

It should be understood that step 202 is not a necessary step; instead, it is an added feature that can be implemented to improve user experience. In embodiments where step 202 does not occur, steps 204 and 206 are no longer needed, though it is contemplated that steps 204 and 206 can nevertheless occur such that recommendations are ready for use in other aspects of the inventive subject matter (e.g., replacement meals can be suggested for users even when no placeholder meal exists in a time slot).

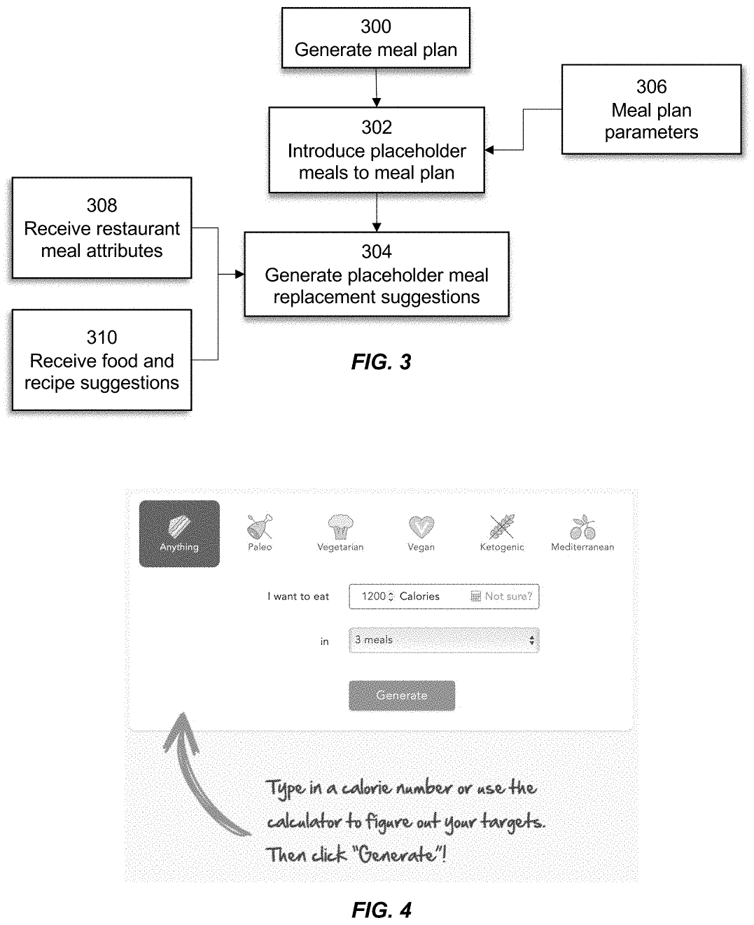

The steps described above regarding FIG. 2 are directed to embodiments where placeholder meals are created at the time a meal plan is generated. In some embodiments, placeholder meals can be added—or substituted—into a meal plan after the meal plan's generation. FIG. 3 shows an embodiment where one or more placeholder meals are introduced after a meal plan has already been generated. For example, an existing meal in a meal plan can be converted into a placeholder meal via user request. In embodiments where a user sets a meal as a placeholder meal after a mean plan's generation, an existing meal in the meal plan can be set by a user via user device to change that meal to a placeholder. The existing meal would thus be made into a non-specific entry having various attributes, which can include a calorie quantity, nutritional values (e.g., fat, carbohydrates, and protein quantities), meal recommendations, recipe recommendations, etc.

Thus, in step 300, a meal plan is generated. Meal plan generation is undertaken as described above, accounting for different meal attributes given by a user via user device. Step 302 describes introducing placeholder meals to the meal plan. Placeholder meal parameters are defined according to step 306, which is analogous to step 204 described above.

Like step 202, above, step 304 is directed to generating placeholder meal replacement recommendations. Restaurant meal attributes and food and recipe suggestions are received by the platform server according to steps 308 and 310, respectively (just like steps 206 and 208, above). Using this received information, the platform server can generate meal replacement suggestions, giving a user some options to substitute a meal in for a placeholder meal. As discussed above, meal replacement suggestions can be based on any attribute that is user-defined or user-selected within the platform server (e.g., dietary restrictions, previously selected meal replacements, pantry ingredients, etc.). Additionally, meal replacement suggestions can also account for a user's location (e.g., GPS, address, user-defined location, etc.), a desired search radius, specified preferred restaurants, and so on. User profile attributes such as nutrition targets, budget, cook time, etc., can also be accounted for when suggesting meal replacements. For non-restaurant suggestions, the platform server can use any or all of the user's profile preferences (e.g., nutrition targets, budget, cook times, etc.).

FIGS. 4-10 show example user interfaces that facilitate user interaction with platform servers of the inventive subject matter. These interfaces can be accessed via, e.g., web browser or software application, and each interface screen shown can be used to either display information sent to a user device by the platform server or to collect information from a user to be sent from the user's device to the platform server. It should be understood that any action on a user device can be transmitted to the platform server in the form of instruction or request for the platform server to take a specified action. Requests or instructions can additionally include information sufficient to carry out the request or instruction (e.g., when a meal is converted into a placeholder meal, the user device transmits a request to convert the meal into a placeholder meal and that request can include placeholder meal parameters). Moreover, any subset of the parameters and information found in FIGS. 4-10 can be collected or displayed—there is no requirement that all parameters and information shown in these figures be collected or displayed in every embodiment. FIG. 4 shows a user interface that a user can access using, e.g., a web browser. The interface allows the user to select a diet type (e.g., anything, paleo, vegetarian, vegan, ketogenic, and Mediterranean) as well as a number of calories per day the user would like to consume and how many meals per day that user would like to consume.

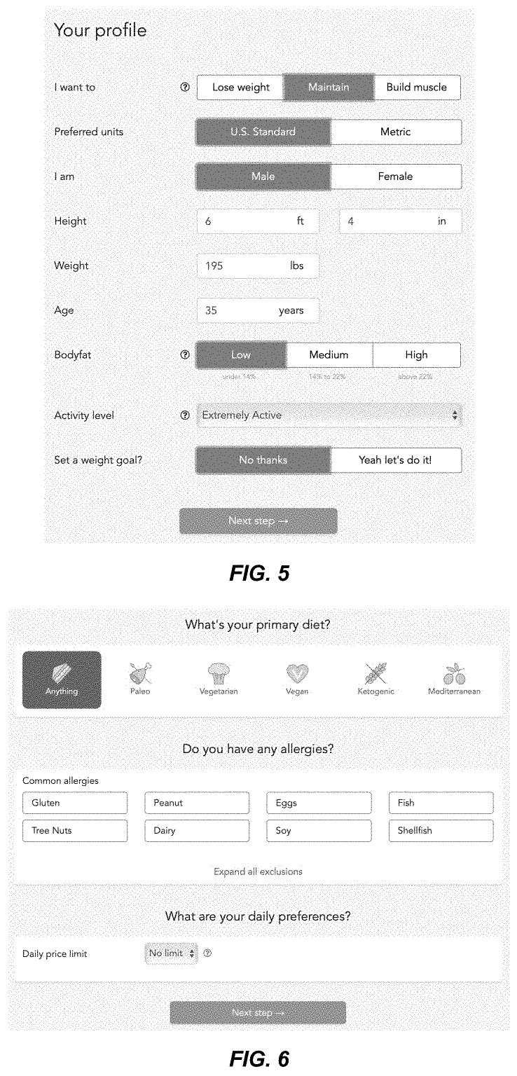

To improve meal plan generation, users can optionally provide information about themselves. FIG. 5 shows an interface allowing users to input body parameters, including, a weight goal, units of measurement, gender, height, current weight, age, a body fat range, activity level, and a weight goal. All these parameters can be used to, e.g., improve meal suggestions. Upon making these selections, the user's device can transmit the collected information to the platform server where it can be stored in a database. All information collected relating to a specific user can be stored in association with a user profile that is stored on or in association with the platform server.

FIG. 6 shows an interface where a user can provide additional diet information to the platform server via user device. For example, a user can input various allergies or other dietary restrictions (e.g., foods that the user would like excluded from meals). Users can also provide price sensitivity information (e.g., no price limit, low price limit, medium price limit, and high price limit, all of which can be based on a user's input on, for example, a lowest and highest price per meal they are willing to tolerate), which can be used to improve meal suggestions by ensuring users are given meal suggestions that match their desired price limitations.

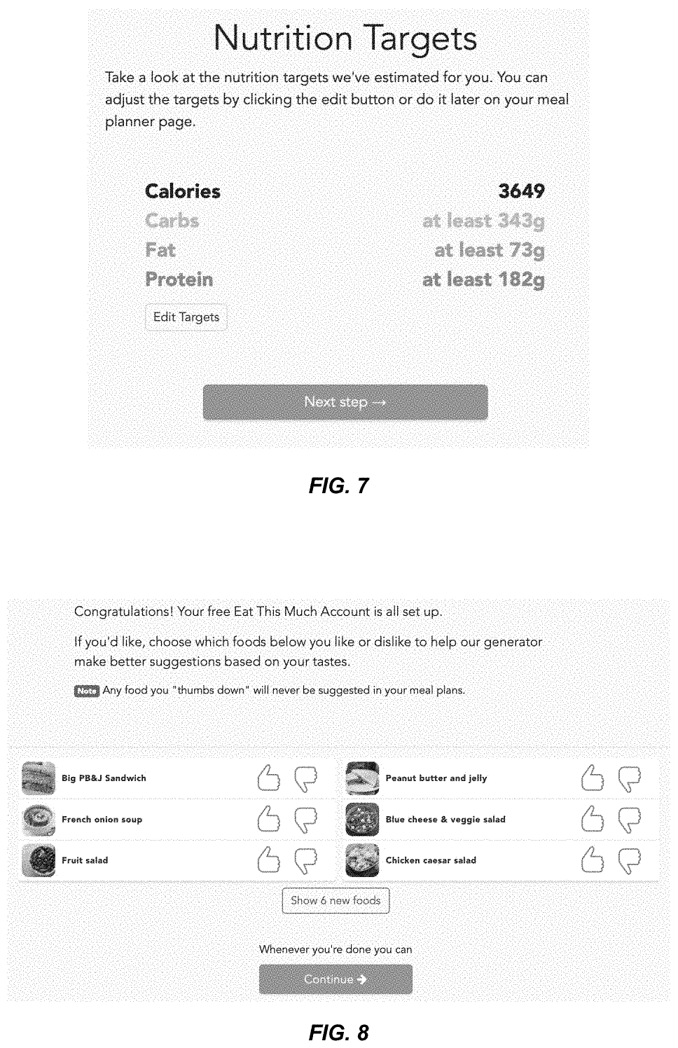

FIG. 7 shows an interface that allows a user to set preferences related to nutrition targets (e.g., quantities of calories, fat, protein, carbohydrates). For example, a diet for the individual described in FIG. 5 has been given the nutritional targets shown in FIG. 7. But users are able to edit those targets as needed, giving the platform server added flexibility to develop customized meal plans. FIG. 8 shows an interface allowing a user to select foods or meals they enjoy. Users can give a simple binary indication of “like” (thumbs up) or “dislike” (thumbs down). This information can be used to improve meal suggestions the platform server generates for that user.

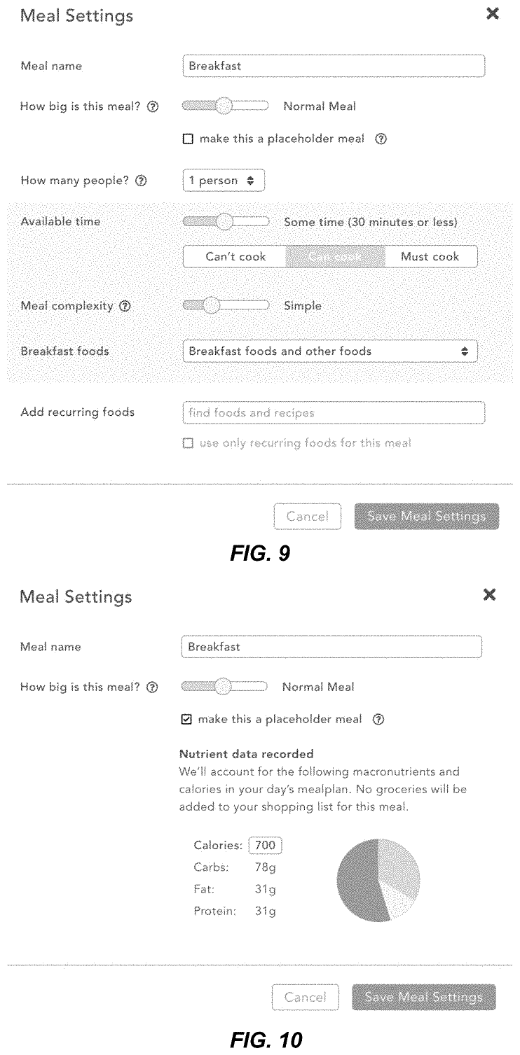

FIG. 9 shows meal parameters for a single meal in a meal plan including an option to make a meal into a placeholder meal. As described above, users can convert one or more meals into placeholder meals in an already generated meal, and the interface shown in FIG. 9 is an example of how that can be accomplished. Meal parameters include a meal name (e.g., breakfast, lunch, dinner, snack), meal size (e.g., small, medium, large), a placeholder flag (e.g., a checkbox or toggle), a number of people the meal is for, an available time (e.g., a slider that allows a user to set an amount of time allotted for the meal, generally between 5 minutes and 2 hours), an indication as to whether the user can prepare the meal (e.g., can good, can't cook, must cook), a meal complexity (e.g., a slider that allows users to move the meal from low to high complexity), a preferred food type (e.g., breakfast food, dinner food, lunch food), and a box for a user to input a food or recipe search query that can be used to input recurring foods for the meal.

FIG. 10 shows how the meal parameters interface from FIG. 9 changes when the placeholder meal box is checked. A placeholder meal does not include as much detail regarding the meal except that the meal should have some number of calories. A placeholder meal, while not specifically defined, allows users to input roughly a number of calories and, in some embodiments, nutritional information such as quantities carbohydrates, fat, and protein. This feature allows users added flexibility and makes services of the inventive subject matter more approachable by giving users an option to, essentially, estimate a meal if they don't have the time or energy to plan something more specific.

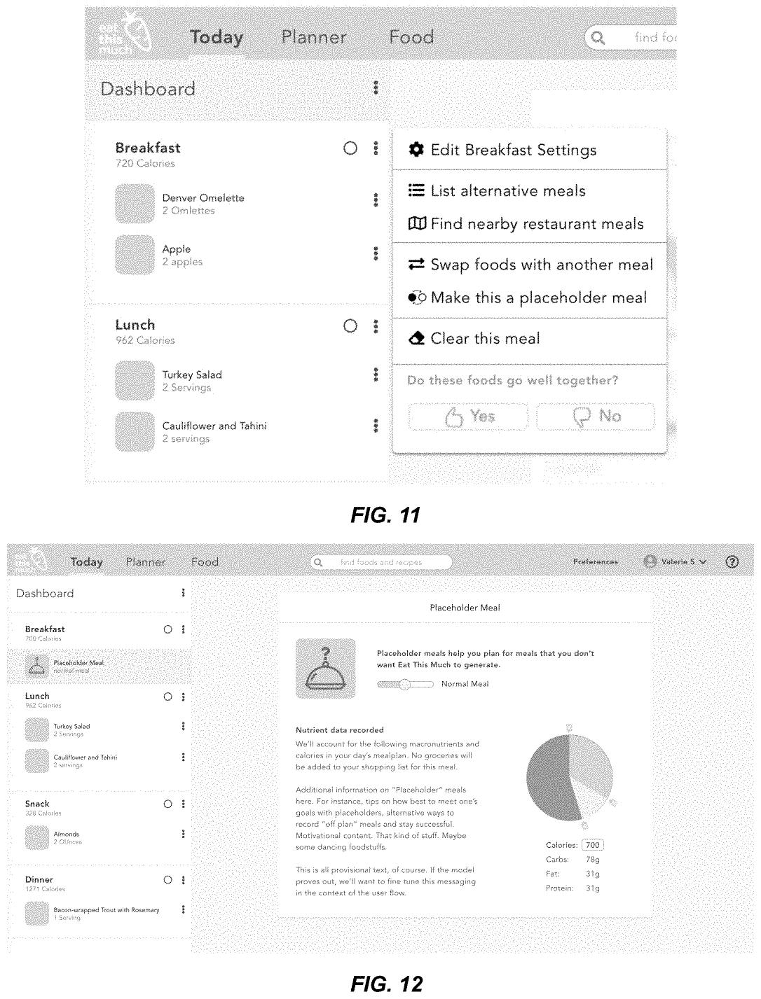

Another example of how a placeholder meal can be set is shown in FIG. 11, which shows an interface that can be accessed via a daily meal planner. FIG. 11 thus shows options for the breakfast entry, including an option to convert the breakfast entry into a placeholder meal. Once a meal has been converted, its daily planner entry can change as shown in FIG. 12. Users are able to adjust meal size from this interface.

Placeholder meals can additionally be set to recur. For example, a user can instruct the platform server to set all Tuesday lunches to a placeholder meal if, for example, that user knows that on Tuesdays they will be going with colleagues to a work lunch every Tuesday and they cannot know with certainty what meal they will have.

When a placeholder meal exists in a meal plan, restaurant and meal suggestions, discussed above, can be provided to a user based on a variety of parameters, including the user's food preferences, nutrition targets, location, restaurant preferences, and so on. Using all or some of those parameters, the platform server can send a list of possible meals (e.g., restaurant meals or meal suggestions) to the user to allow the user to select a meal. In situations where the platform server is providing suggestions for a placeholder meal, once the user choose a suggested meal, the placeholder meal is replaced by the selection for that meal slot (e.g., if the placeholder meal is recurring, only one placeholder is replaced, and the remaining placeholder meals in the future are in affected). In embodiments where a list of suggested meals includes one or more meals at one or more restaurants, the platform server can send the user additional information including restaurant location and hours of operation.

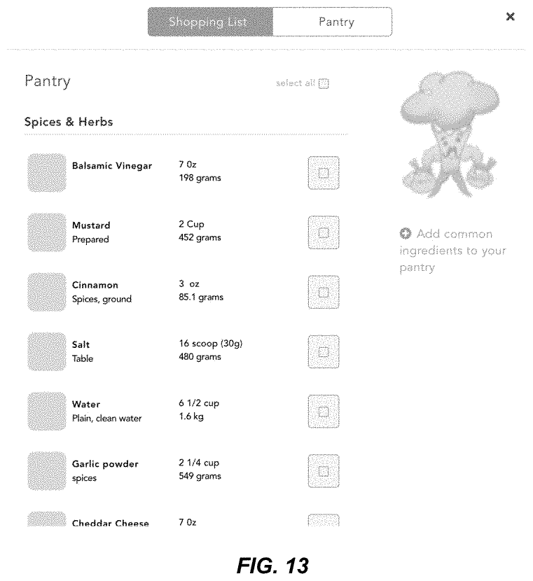

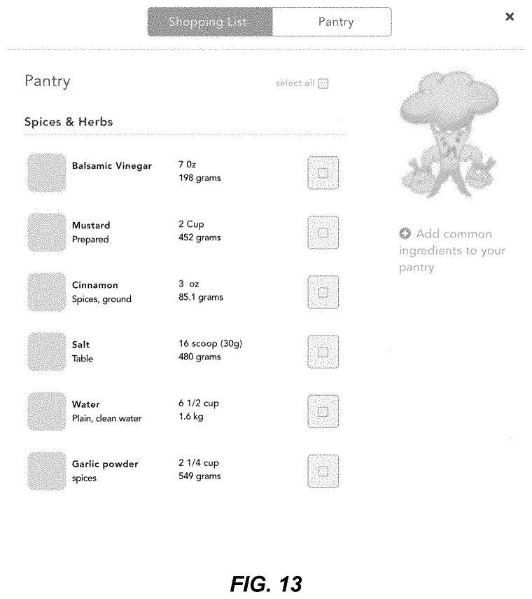

The platform server can make meal suggestions based on still further information in addition to all those parameters discussed above (e.g., allergies, budget, nutrition targets, available cook time, etc.). In some embodiments, users can specify ingredient and food availability to the platform server to maintain a virtual “pantry” that mirrors the ingredient and food available of that user's own pantry (e.g., at home, at work, or the like). Users can additionally specify how much of a particular food or ingredient they have. An example interface showing the virtual pantry is shown in FIG. 13. That information can help the platform server develop better meal suggestions for the user by creating suggestions based on the user's available ingredients and foods. In some embodiments, displaying meal suggestions that come from a user's currently available foods and ingredients can be prioritized over other meal suggestions. If a meal is not set as a placeholder and the user does not select a suggested meal, that meal slot can remain empty, though empty meal slots can occur for a variety of reasons not limited to those discussed here.

Thus, specific systems and methods directed to meal plan generation that incorporate placeholder meals have been disclosed. It should be apparent, however, to those skilled in the art that many more modifications besides those already described are possible without departing from the inventive concepts in this application. The inventive subject matter, therefore, is not to be restricted except in the spirit of the disclosure. Moreover, in interpreting the disclosure all terms should be interpreted in the broadest possible manner consistent with the context. In particular the terms “comprises” and “comprising” should be interpreted as referring to the elements, components, or steps in a non-exclusive manner, indicating that the referenced elements, components, or steps can be present, or utilized, or combined with other elements, components, or steps that are not expressly referenced.

What is claimed is:

1. A method of generating a meal plan comprising:
receiving, at a platform server from a user device, meal plan parameters comprising placeholder meal parameters for at least one meal;
generating, by the platform server, the meal plan according to the meal plan parameters, the meal plan having a first meal and a second meal;
wherein the second meal comprises a placeholder meal defined at least in part according to the placeholder meal parameters; and
making the meal plan accessible to the user device.

2. The method of claim 1, wherein at least one placeholder meal parameter of the placeholder meal parameters comprises a specified meal slot, wherein the specified meal slot is defined by a user via the user device.

3. The method of claim 1, wherein the placeholder meal parameters comprise quantities for calories, fat, protein, and carbohydrates.

4. The method of claim 1, wherein the meal plan parameters comprise daily quantities for calories, fat, protein, and carbohydrates.

5. The method of claim 4, wherein quantities of calories, fat, protein, and carbohydrates of the placeholder meal are determined according to the daily quantities for calories, fat, protein, and carbohydrates.

6. A method of generating a meal plan comprising:
receiving, at a platform server from a user device, meal plan parameters;
generating, by the platform server, the meal plan according to the meal plan parameters, the meal plan having a first meal and a second meal;
making the meal plan accessible to the user device;
receiving, at the platform server from the user device, instructions to convert the second meal to a placeholder meal comprising placeholder meal parameters;
generating, by the platform server, an updated meal plan comprising the placeholder meal; and
making the updated meal plan accessible to the user device.

7. The method of claim 6, wherein the instructions comprise a specified meal slot, wherein the specified meal slot is defined by the user via the user device and wherein the specified meal slot indicates the second meal should be replaced with the placeholder meal.

8. The method of claim 6, wherein the placeholder meal parameters comprise quantities for calories, fat, protein, and carbohydrates.

9. The method of claim 6, wherein the meal plan parameters comprise daily quantities for calories, fat, protein, and carbohydrates.

10. The method of claim 9, wherein quantities of calories, fat, protein, and carbohydrates of the placeholder meal are determined according to the daily quantities for calories, fat, protein, and carbohydrates.

11. A method of generating a meal plan comprising:
receiving, at a platform server from a user device, meal plan parameters comprising a first set of meal parameters;
generating, by the platform server, the meal plan according to the meal plan parameters, the meal plan having at least one meal generated according to the first set of meal parameters;
receiving, at the platform server from the user device, updated meal plan parameters comprising a second set of meal parameters;
generating, by the platform server, an updated meal plan according to the updated meal plan parameters, the updated meal plan comprising the at least one meal and a second meal generated according to the second set of meal parameters, wherein the second meal is a placeholder meal; and
making the updated meal plan accessible to the user device.
